SEQUENCE LISTING (5 sequences)

SEQ ID NO: 1 - protein, 37 aa, synthetic construct
  SITE 37: note = Amidated residue
KCNTATCATQRLAEFLRHSSNNFGPILPPTNVGSNTP

SEQ ID NO: 2 - protein, 37 aa, synthetic construct
  SITE 37: note = Amidated trans-hydroxyproline (trans-Hyp)
KCNTATCATQRLADFLRHSSNNLKPILPPTNVGSNTX

SEQ ID NO: 3 - protein, 32 aa, synthetic construct
  SITE 32: note = Amidated residue
ASQLSTAVLGRLSQELHRLQDYPRTDVGSGSP

SEQ ID NO: 4 - protein, 32 aa, synthetic construct
  SITE 32: note = Amidated residue
ASQLSTAVLGRLSDELHRLQDYPRTDVGSGSP

SEQ ID NO: 5 - protein, 32 aa, synthetic construct
  SITE 32: note = Amidated residue
CSNLSTCVLGKLSQELHKLQTYPRTNTGSGTP

(19) United States
(12) Patent Application Publication
(10) Pub. No.: US 2026/0356892 A1
(43) Pub. Date: Nov. 12, 2026

(54) POLYPEPTIDE DERIVATIVE FOR ORAL DELIVERY

(71) Applicants: Hangzhou Sciwind Biosciences Co., Ltd., Hangzhou (CN); Sciwind Biosciences (Beijing) Co., Ltd., Beijing (CN)

(72) Inventors: Yan LI, Beijing (CN); Sujuan HAO, Beijing (CN); Zhaoying LI, Beijing (CN); Hai PAN, Beijing (CN); Haixia ZOU, Beijing (CN); Xinle WU, Beijing (CN)

(21) Appl. No.: 19/718,896

(22) Filed: Jun. 24, 2026

Related U.S. Application Data
(63) Continuation of application No. 19/387,410, filed on Nov. 12, 2025.
Application No. 19/387,410 is a continuation of application No. PCT/CN2024/095763, filed on May 28, 2024.

(30) Foreign Application Priority Data
May 29, 2023 (CN) 202310619176.4

(51) Int. Cl. C07K 14/00; A61K 9/00; A61K 9/20; A61K 38/00; A61P 3/04; C07K 1/107

(52) U.S. Cl. CPC C07K 14/001; A61K 9/0053; A61K 9/2013; A61P 3/04; C07K 1/1075; A61K 38/00

(57) ABSTRACT

Provided herein are use of a fatty acid-containing side chain in improving activity, activity selectivity, and oral availability of a polypeptide derivative, a method for achieving the use, and a polypeptide derivative modified by the side chain.

### CROSS-REFERENCE TO RELATED APPLICATIONS

This application is a continuation of U.S. patent application Ser. No. 19/387,410, filed on Nov. 12, 2025, which is a continuation of International Application No. PCT/CN2024/095763, filed on May 28, 2024, which claims the benefit of China Patent Application No. 202310619176.4, filed on May 29, 2023, all of which are incorporated by reference herein in their entirety.

### TECHNICAL FIELD

The present application relates to the technical field of biomedicine or biopharmaceuticals, specifically, a polypeptide or a derivative thereof for oral delivery, a composition of the polypeptide or the derivative thereof for oral delivery, and use of them for treating and/or preventing metabolism associated diseases.

### INCORPORATION BY REFERENCE OF SEQUENCE LISTING

The instant application contains a Sequence Listing which has been submitted electronically in XML format and is hereby incorporated by reference in its entirety. Said XML copy, created on Jul. 14, 2026, is named 70304-706_302_SL.xml and is 7,548 bytes in size.

### BACKGROUND

Polypeptides/proteins are not only indispensable elements as an essential cellular component in an organism, but also play important roles in maintaining normal physiological activities of the organism as major participants of intercellular or inter-tissue signaling. Since bovine insulin and porcine insulin were used for treatment of diabetes for the first time in the early 2000s, more and more polypeptides/proteins have been produced and applied in the field of human pharmaceuticals as modern technologies of molecular biology and organic chemistry advance. Polypeptide/protein-based drugs have distinct advantages in treatment of diseases including cancer, diabetes, cardiocerebrovascular diseases, autoimmune diseases, and metabolic disorders over their counterparts of other types due to their high specificity for targets as well as desirable safety and tolerance. However, a large proportion of polypeptide/protein-based drugs with therapeutic activity have only modest oral bioavailability due to limitations imposed by the mechanism of absorption of polypeptides/proteins in the human body, and thus need to be administered by injections or other routes for treating diseases, which hinders the development of polypeptide/protein-based drugs and brings about additional pain and inconvenience for patients receiving the treatment.

Many attempts have been made to enhance the effect of orally administered polypeptide-based drugs in order to fix that deficiency in oral polypeptides. For example, Fiona McCartney et al. (Journal of Controlled Release, Volume 310, 28 Sep. 2019, Pages 115-126) disclosed Labrasol® ALF (Labrasol®), a non-ionic surfactant excipient for use in enhancing bioavailability of insulin in the intestine; P. Uhl et al. (Nanomedicine: Nanotechnology, Biology and Medicine, Volume 24, February 2020, 102132) discovered that coating of PLA nanoparticles with cyclic, arginine-rich cell penetrating peptides enables oral delivery of liraglutide; and Vivek Gupta et al. (Journal of Controlled Release, Volume 172, Issue 3, 28 Dec. 2013, Pages 753-762) disclosed preparation of mucoadhesive devices by compressing a polymeric matrix containing carbopol, pectin and sodium carboxymethylcellulose (1:1:2), and demonstrated its effect of improving salmon calcitonin (sCT) delivery in vivo.

However, current polypeptide-based drugs still have problems such as poor oral efficacy, low bioavailability, unpredictable delivery effects of corresponding oral administration regimens for the polypeptide molecules, and poor versatility.

### SUMMARY OF THE INVENTION

Accordingly, the present application provides for a polypeptide derivative with increased activity and stability as well as an improved effect and bioavailability for oral delivery, which is capable of effectively treat and/or prevent metabolism associated diseases. Specifically, the present application is directed to a technical solution comprising the following embodiments:
1. Use of a fatty acid-containing side chain in improving activity selectivity of a polypeptide derivative, wherein the polypeptide derivative is formed by modification of a polypeptide molecule comprising the fatty acid-containing side chain, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
2. Use of a fatty acid-containing side chain in improving activity of a polypeptide derivative, wherein the polypeptide derivative is formed by modification of a polypeptide molecule comprising the fatty acid-containing side chain, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
3. Use of a fatty acid-containing side chain in improving oral bioavailability of a polypeptide derivative, wherein the polypeptide derivative is formed by modification of a polypeptide molecule comprising the fatty acid-containing side chain, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
4. A method of improving activity selectivity of a polypeptide derivative, comprising modifying a polypeptide molecule comprising a fatty acid-containing side chain to form the polypeptide derivative, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
5. A method of improving activity of a polypeptide derivative, comprising modifying a polypeptide molecule comprising a fatty acid-containing side chain to form the polypeptide derivative, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
6. A method of improving oral bioavailability of a polypeptide derivative, comprising modifying a polypeptide molecule comprising a fatty acid-containing side chain to form the polypeptide derivative, wherein the fatty acid-containing side chain is in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
7. The use of any one of embodiments 1-3 or the method of any one of embodiments 4-6, wherein Z1 is a C16, C17, C18, C19 or C20 fatty diacid.
8. The use or the method of any one of embodiments 1-7, wherein Z2 is selected from any one of γGlu, βAsp, γGlu, Inp, and Trx, preferably βAsp or γGlu.
9. The use or the method of any one of embodiments 1-8, wherein n is greater than 0, preferably 1-10, more preferably 2-8, and further preferably, n=2.
10. The use or the method of any one of embodiments 1-9, wherein Z1 is a C16, C18 or C20 fatty diacid.
11. The use or the method of any one of embodiments 1-10, wherein Z2 is Inp or Trx; and n is greater than 0.
12. The use or the method of any one of embodiments 1-11, wherein Z1 is a C16, C17, C18, C19 or C20 fatty diacid; Z2 is γGlu; and n=2.
13. The use or the method of any one of embodiments 1-12, wherein Z1, Z2, and Z3 are linked via amide linkages.
14. The use or the method of any one of embodiments 1-13, wherein the modification of the side chain is on 1-10 amino acids, preferably 1-3 amino acids, and more preferably 1 amino acid, of the polypeptide.
15. The use or the method of any one of embodiments 1-14, wherein the modification of the side chain is at an amino acid at the N terminus or any middle position in the sequence of the polypeptide, wherein the amino acid is preferably selected from glutamic acid, aspartic acid, lysine, glutamine, or asparagine, and is further preferably lysine.
16. The use or the method of any one of embodiments 1-15, wherein the polypeptide is an agonistic or antagonistic, natural or artificially synthetic polypeptide molecule, preferably selected from a GLP-1 receptor agonist, a GIP receptor antagonist, an insulin receptor agonist, a heparin receptor agonist, somatotrophin, an interferon receptor agonist, an interleukin receptor agonist, a follicle-stimulating hormone receptor agonist, a gonadotropin receptor agonist, an erythropoietin receptor agonist, a Peptide YY (PYY) receptor agonist, an oxygenation modulator receptor agonist, a glucagon-like peptide-2 (GLP-2) receptor agonist, a calcitonin receptor agonist, a parathyroid hormone (PTH) receptor agonist, a ghrelin receptor agonist, an endocannabinoid receptor agonist, a leptin receptor agonist, a serotonin receptor agonist, a fibroblast growth factor 21 (FGF21) receptor agonist, a cholecystokinin (CCK) receptor agonist, an oxyntomodulin receptor agonist, or a glucagon receptor agonist, preferably an amylin receptor agonist, and further preferably an amylin or calcitonin analog.
17. The use or the method of any one of embodiments 1-16, wherein the polypeptide comprises an amino acid sequence of ASQLS TAVLG RLSDE LHRLQ DYPRT DVGSG SP-NH₂ (SEQ ID NO: 4), KCNTATCATQ RLADFLRHSS NNLKPILPPT NVGSNT-trans-Hyp-NH₂ (SEQ ID NO: 2), or KCNTATCATQ RLAEFLRHSS NNFGPILPPT NVGSNTP-NH₂ (SEQ ID NO: 1), and wherein
preferably, the polypeptide derivative is selected from any one or more of N1 or N2 or M1-M30, or preferably, the polypeptide derivative is selected from any one or more of N1, N2, N3, or M1-M30.
18. The use of embodiment 1 or any one of embodiments 7-17 or the method of embodiment 4 or any one of embodiments 7-17, wherein the improving the activity selectivity of the polypeptide derivative is improving the activity of the polypeptide derivative against an amylin receptor preferentially over a calcitonin receptor, preferably is improving a ratio of the activity of the polypeptide derivative against an amylin receptor to that against a calcitonin receptor, and further preferably comprises improving the activity of the polypeptide derivative against an amylin receptor.
19. The use of embodiment 1 or any one of embodiments 7-18 or the method of embodiment 4 or any one of embodiments 7-18, wherein the improving the activity selectivity of the polypeptide derivative is improving the ratio of the activity against an amylin receptor to that against a calcitonin receptor in a cell by more than 1.5 folds, preferably more than 2 folds, further preferably more than 5 folds, and still further preferably more than 10 folds.
20. The use or the method of embodiment 18 or 19, wherein the activity is agonistic activity.
21. The use of embodiment 1 or any one of embodiments 7-20 or the method of embodiment 4 or any one of embodiments 7-20, wherein the polypeptide derivative is an amylin receptor agonist, preferably is an amylin or calcitonin derivative, and more preferably comprises an amino acid sequence of ASQLS TAVLG RLSDE LHRLQ DYPRT DVGSG SP-NH₂(SEQ ID NO: 4), or further preferably comprises an amino acid sequence of KCNTATCATQ RLADFLRHSS NNLKPILPPT NVGSNT-trans-Hyp-NH₂ (SEQ ID NO: 2).
22. The use of embodiment 2 or any one of embodiments 7-17 or the method of embodiment 5 or any one of embodiments 7-17, wherein the activity of the polypeptide derivative is activity in losing weight and/or reducing food intake.
23. The use of embodiment 2 or any one of embodiments 7-17 or the method of embodiment 5 or any one of embodiments 7-17, wherein the improving the activity of the polypeptide derivative is improving the activity of the polypeptide derivative against an amylin receptor and a calcitonin receptor in a cell.
24. The use of embodiment 2 or any one of embodiments 7-17 and 22-23 or the method of embodiment 5 or any one of embodiments 7-17 and 22-23, wherein the polypeptide derivative is an amylin receptor agonist, preferably is an amylin or calcitonin derivative, and more preferably comprises an amino acid sequence of KCNTATCATQ RLADFLRHSS NNLKPILPPT NVGSNT-trans-Hyp-NH₂ (SEQ ID NO: 2) or KCNTATCATQ RLAEFLRHSS NNFGPILPPT NVGSNTP-NH₂(SEQ ID NO: 1).
25. The use of embodiment 3 or any one of embodiments 7-17 or the method of embodiment 6 or any one of embodiments 7-17, wherein the polypeptide derivative with the fatty acid-containing side chain is co-administered with one or more oral delivery agents, preferably a salt of N-[8-(2-hydroxybenzoyl)amino]caprylic acid (NAC), and more preferably PNAC.
26. The use of embodiment 3 or any one of embodiments 7-17 or the method of embodiment 6 or any one of embodiments 7-17, wherein the improving the oral bioavailability of the polypeptide derivative is achieving higher oral bioavailability than a polypeptide molecule of Cagrilintide.
27. The use of embodiment 3 or any one of embodiments 7-17 or the method of embodiment 6 or any one of embodiments 7-17, wherein the polypeptide derivative is an amylin receptor agonist, preferably is an amylin or calcitonin derivative, and more preferably comprises an amino acid sequence of KCNTATCATQ RLADFLRHSS NNLKPILPPT NVGSNT-trans-Hyp-NH₂ (SEQ ID NO: 2) or KCNTATCATQ RLAEFLRHSS NNFGPILPPT NVGSNTP-NH₂(SEQ ID NO: 1).
28. A derivative of a polypeptide, comprising the polypeptide and a side chain linked thereto in the form of Z1+Z2+Z3, wherein Z1 is a C16-C22 fatty diacid;
   Z2 is selected from any one of γGlu, αGlu, βAsp, αAsp, Inp, and Trx, or is absent; and
   Z3 is n AEEA(s), wherein n≥0.
29. The derivative of embodiment 28, wherein Z1 is a C16, C17, C18, C19 or C20 fatty diacid.
30. The derivative of embodiment 28 or 29, wherein Z2 is selected from any one of γGlu, Inp, and Trx, or is absent.
31. The derivative of any one of embodiments 28-30, wherein n is greater than 0.
32. The derivative of any one of embodiments 28-31, wherein Z2 is Inp or Trx; and n is greater than 0.
33. The derivative of any one of embodiments 28-32, wherein Z2 is absent; and n is greater than 0.
34. The derivative of any one of embodiments 28-33, wherein Z2 is γGlu; and n=2.
35. The derivative of any one of embodiments 28-34, wherein the polypeptide is an amylin receptor agonist; preferably, the derivative of the polypeptide is an amylin or calcitonin derivative; and more preferably, the polypeptide comprises an amino acid sequence of ASQLS TAVLG RLSDE LHRLQ DYPRT DVGSG SP-NH₂(SEQ ID NO: 4), KCNTATCATQ RLADFLRHSS NNLKPILPPT NVGSNT-trans-Hyp-NH₂ (SEQ ID NO: 2), or KCNTATCATQ RLAEFLRHSS NNFGPILPPT NVGSNTP-NH₂(SEQ ID NO: 1), and wherein
preferably, the derivative of the polypeptide is selected from any one or more of N1 or N2 or M1-M30, or preferably, the derivative of the polypeptide is selected from any one or more of N1, N2, N3, or M1-M30.
36. A pharmaceutical composition comprising the derivative of any one of embodiments 28-35 or a pharmaceutically acceptable salt thereof, and a pharmaceutically acceptable excipient, wherein preferably, the composition comprises one or more oral delivery agents, preferably a salt of N-[8-(2-hydroxybenzoyl)amino]caprylic acid (NAC), and further preferably PNAC.
37. Use of the derivative of any one of embodiments 28-35 or a pharmaceutically acceptable salt thereof or the composition of embodiment 36 in the manufacture of a polypeptide-based medicament; wherein preferably, the polypeptide-based medicament is used for preventing and/or treating overweight and/or obesity and/or type I or type II diabetes and/or osteoporosis and/or neuropathic pain.
38. The use of embodiment 37, wherein the polypeptide-based medicament is an oral medicament; and preferably, the oral medicament comprises one or more oral delivery agents, preferably a salt of N-[8-(2-hydroxybenzoyl)amino]caprylic acid (NAC), and further preferably PNAC.
39. Use of the derivative of any one of embodiments 28-35 or a pharmaceutically acceptable salt thereof or the pharmaceutical composition of embodiment 36 in reducing food intake.
40. A method of preventing and/or treating overweight and/or obesity and/or type I or type II diabetes and/or osteoporosis and/or neuropathic pain, comprising administering to a subject a prophylactically or therapeutically effective amount of the derivative of any one of embodiments 28-35 or a pharmaceutically acceptable salt thereof or the pharmaceutical composition of embodiment 36.
41. A method of reducing food intake, comprising administering to a subject an effective amount of the derivative of any one of embodiments 28-35 or a pharmaceutically acceptable salt thereof or the pharmaceutical composition of embodiment 36.

### Technical Effect

The polypeptide derivatives of the present application have high activity in cells, strong potency or effectiveness on amylin and calcitonin receptors, and stable characteristics and properties including concentration and purity with little change after storage for a long period of time.

The polypeptide derivatives of the present application produce prominent effects on losing weight and reducing food intake and attain enhanced efficacy and bioavailability as oral drugs.

### BRIEF DESCRIPTION OF THE DRAWINGS

The drawings are intended for a better understanding rather than any undue limitation of the present application, in which:

FIG. 1 is a trend chart showing the effect of the polypeptide derivatives on weight change of rats;

FIG. 2 is a trend chart showing the effect of the polypeptide derivatives on accumulated food intake of rats;

FIG. 3 is a trend chart showing the effect of some of the polypeptide derivatives on weight change of rats;

FIG. 4 is a trend chart showing the effect of some of the polypeptide derivatives on accumulated food intake of rats;

FIG. 5 shows the plasma level of polypeptide derivatives D1 and N2; and

FIG. 6 shows the plasma level of some of the polypeptide derivatives as oral tablets.

### DETAILED DESCRIPTION

Illustrative examples of the present application are described as below, in which various details of the examples in the present application are included for ease of understanding and should be construed as illustrative only. Therefore, it is to be realized by those of ordinary skill in the art that many alterations and modifications may be made to the examples described here without departing from the scope and spirit of the present application. The following description also omits depiction of well known functions and structures for clarity and conciseness, and terms about technology and science used in this specification have the same meaning as generally understood by those skilled in the art, unless otherwise defined.

The terms “polypeptide”, “peptide”, and “protein” are used interchangeably herein and refer to an amino acid polymer of any length. The polymer can be linear or branched and may comprise naturally occurring amino acids among which non-naturally occurring ones may be interspersed.

The term “N terminus” refers to the N or amino terminal of a linear polypeptide consisting of a plurality of amino acids linked via peptide bonds and containing a free a amino group (—NH₂) and a free a carboxyl group (—COOH) that, when unmodified, are unbonded in respective amino acids at both ends, i.e., the terminus harboring an unbonded, free α-NH₂ in the polypeptide. A modification site at the N terminus or an N terminal modification refers to making a modification to an amino acid at the N terminus of the polypeptide.

The term “derivative” herein refers to a product resulting from a modification of a functional group (e.g., an amino acid residue) in a biomacromolecule (e.g., a polypeptide or protein) with a certain compound or molecule. The modification includes but is not limited to acylation, amidation, esterification, and thioesterification.

The term “activity selectivity” of a polypeptide or a polypeptide derivative herein refers to an attribute of the polypeptide or polypeptide derivative where it has a stronger effect on one of the receptors it acts on than another. In some embodiments of the present application, the activity refers to agonistic activity.

In some embodiments of the present application, the activity selectivity may further refer to preferentially improved activity of the polypeptide derivative against an amylin receptor over a calcitonin receptor. Preferably, the activity selectivity refers to an improved ratio of activity of the polypeptide derivative against an amylin receptor to that against a calcitonin receptor.

A pharmaceutical composition herein may be used for the treatment of diseases or for cell culture assays in vitro. When used for the treatment of diseases, the term “pharmaceutical composition” generally refers to a unit dosage form and may be prepared by any of the methods well known in the field of pharmaceutics. The methods used include a step of blending the active ingredients with excipients that are one or more auxiliary ingredients. Typically, a composition is prepared by uniformly and adequately blending active compounds with a liquid excipient, a fine solid excipient or both.

Being “pharmaceutically acceptable” herein refers to an attribute of a substance or composition that has to be chemically and/or toxicologically compatible with other ingredients contained in the formulation and/or mammals treated thereby.

The term “pharmaceutically acceptable excipient” herein may include any solvent, solid excipient, diluent or other liquid excipients or the like suitable for particular target dosage forms. Use of any conventional excipients is also within the scope contemplated in the present application, except to the extent that they are incompatible with the compounds of the present application, for example, result in any undesirable biological effects or interact with any other components of the pharmaceutically acceptable composition in a deleterious way.

The term “treatment” herein refers to the attainment of a desired pharmacological and/or physiological effect. The effect can be prophylactic in terms of completely or partially preventing a disease or a symptom thereof, and/or can be therapeutic in terms of completely or partially curing a disease and/or an adverse effect attributable to the disease. The “treatment” used herein encompasses, with respect to a disease in a mammal, particularly a human, (a) preventing occurrence of a disease or condition in an individual susceptible to but not diagnosed with the disease; (b) inhibiting a disease, for example, impeding the development of the disease; or (c) alleviating a disease, for example, relieving symptoms related to the disease. The “treatment” used herein encompasses any administration of a medicament or compound to an individual to treat, cure, alleviate, ameliorate, palliate or inhibit a disease of the individual, including but not limited to giving a medicament comprising the compound described herein to an individual in need thereof.

The term “prevention” herein refers to lowering the possibility of occurrence (or recurrence) of a disease, condition, or disorder or related symptoms (e.g., cancer).

### ABBREVIATIONS

Corresponding names or structures of some abbreviations used in the examples of the present application are as follows

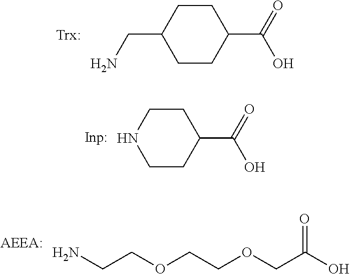

AA: Amino Acid; Boc: t-Butyloxy carbonyl; DCM: dichloromethane; DMF: N,N-Dimethyl formamide; DIEA: N,N-Diisopropylethylamine; EDT: 1,2-Ethanedithiol; Fmoc: 9-fluorenylmethyloxycarbonyl; OtBu: t-butyl ester; Pbf: 2,2,4,6,7-Pentamethyldihydrobenzofuran-5-sulfonyl chloride; Pip: Piperidine; TBTU: 0-(Benzotriazol-1-yl)-N,N,N′,N′,-tetramethyluronium Tetrafluoroborate; tBu: tertiary butyl; TFA: Trifluoroacetic acid; TIS: Triisopropylsilane; Trt: Triphenylmethyl; αGlu: α-glutamic acid; γGlu: γ-glutamic acid; αAsp: α-aspartic acid; and βAsp: β-aspartic acid.

### EXAMPLES

### Example 1 Preparation of Polypeptide Derivatives

Polypeptides derivatives prepared in the present application are shown in Table 2.

Target products in Table 2 were prepared by solid phase organic synthesis, specifically, solid-phase peptide synthesis (SPPS) with Fmoc-protected amino acids, followed by cleavage, oxidization, and purification.

Taking the compound D1 (Cagrilintide) as an example, which has the structure of (SEQ ID NO: 1):

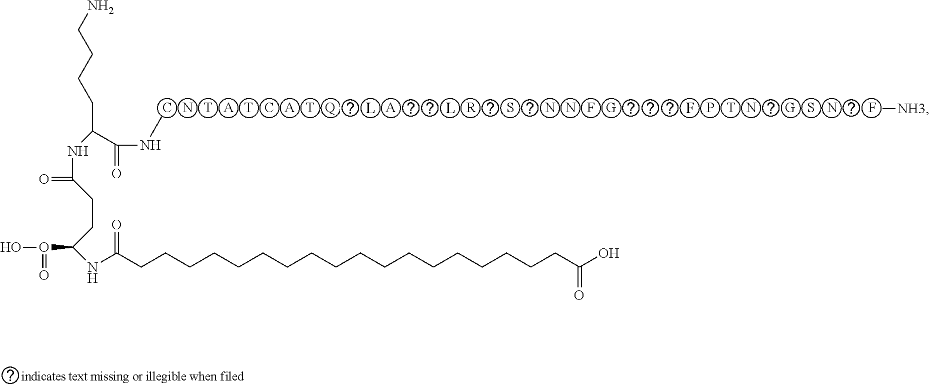

the synthesis process was as below:

### 1.1 Solid-Phase Synthesis

Using Fmoc-Linker MBHA Resin S=0.32 mmol/g and employing Fmoc/tBu processing, amino acids were linked by sequential condensation from the C terminus to the N terminus (from right to left) as per the sequence of the aforesaid peptide in accordance with the method provided in Table 1.

TABLE 1
Listing of synthesis procedures

| | | Number of | duration |
|---|---|---|---|
| No. | Solvent | times | (min/time) |
| 1 | 20% Pip/DMF | 1 | 10 |
| 2 | 20% Pip/DMF | 1 | 5 |
| 3 | DMF | 5 | 1 |
| 4 | Sampling for colorimetric testing | | |
| 5 | AA/DIEA/TBTU | 1 | 60 |
| 6 | DMF | 4 | 1 |

| | |
|---|---|
| 7 | Sampling for colorimetric testing |

Notes
Repeating 1-7 till the end of the linear peptide synthesis

The following amino acids were coupled in order:
A-01 Fmoc-Pro-OH, A-02 Fmoc-Thr(tBu)—OH, A-03 Fmoc-Asn(Trt)-OH, A-04 Fmoc-Ser(tBu)-OH, A-05 Fmoc-Gly-OH, A-06 Fmoc-Val-OH, A-07 Fmoc-Asn(Trt)-OH, A-08 Fmoc-Thr(tBu)-OH, A-09 Fmoc-Pro-OH, A-10 Fmoc-Pro-OH, A-11 Fmoc-Leu-OH, A-12 Fmoc-Ile-OH, A-13 Fmoc-Pro-OH, A-14 Fmoc-Gly-OH, A-15 Fmoc-Phe-OH, A-16 Fmoc-Asn(Trt)-OH, A-17 Fmoc-Asn(Trt)-OH, A-18 Fmoc-Ser(tBu)-OH, A-19 Fmoc-Ser(tBu)-OH, A-20 Fmoc-His(Trt)-OH, A-21 Fmoc-Arg(Pbf)-OH, A-22 Fmoc-Leu-OH, A-23 Fmoc-Phe-OH, A-24 Fmoc-Glu(OtBu)-OH, A-25 Fmoc-Ala-OH, A-26 Fmoc-Leu-OH, A-27 Fmoc-Arg(Pbf)-OH, A-28 Fmoc-Gln(Trt)-OH, A-29 Fmoc-Thr(tBu)-OH, A-30 Fmoc-Ala-OH, A-31 Fmoc-Cys(Trt)-OH, A-32 Fmoc-Thr(tBu)-OH, A-33 Fmoc-Ala-OH, A-34 Fmoc-Thr(tBu)-OH, A-35 Fmoc-Asn(Trt)-OH, A-36 Fmoc-Cys(Trt)-OH, A-37 Fmoc-Lys(Boc)-OH, A-38 Fmoc-Glu-otbu, and A-39 C20 diacid.

Finally, a polypeptide derivative-on-resin was formed.

The polypeptide derivative-on-resin was washed, transferred out, and dried to a constant weight for cleavage.

### 1.2 Cleavage

The cleavage reagent was used in an amount of 10 mL±2 mL per 1 g of the polypeptide derivative-on-resin. For cleavage, H₂O, TFA, EDT, and TIS composing the required cleavage reagent were sequentially added to a cleavage reaction flask in a ratio of TFA:H₂O:EDT:TIS=95:1:2:2 and were controlled at a temperature of 0-10 °C. The cleavage reagent was added to the resin under stirring, and after the temperature of the system became stable, the reactants were stirred for another 2.5 hours under a controlled temperature of 25-30 °C. The cleavage solution was filtered, and allowed for precipitation using glacial diethyl ether of a 5× volume of the liquid. The precipitates were filtered, washed 3 times with glacial diethyl ether of a 3× volume of the liquid, and then dried under reduced pressure at room temperature to yield a solid crude.

### 1.3 Oxidation

The crude was finely ground, and was slowly added, with stirring, to purified water, while an aqueous solution of acetonitrile was added dropwise. After the crude was added and completely dissolved, a methanol solution of iodine was added and stirred for half an hour.

### 1.4 Purification and Lyophilization

The oxidated liquid as described above was filtered with a 0.45 μm microporous filtering membrane. The crude was separated and purified using a column prepared by C-18 column packing material with a suitable gradient at ambient temperature, and then the target product was collected, detected and analyzed, and sorted. A purity ≥90% was required. Substandard target products were collected, and separated and purified again with a suitable gradient, to achieve qualified liquid peaks. The qualified liquid samples as mentioned above were lyophilized under reduced pressure to yield lyophilized powder of the refined polypeptide derivative.

Polypeptide derivatives prepared using a similar method to that described in Example 1 of the present application are shown in Table 2 and are distinguished from each other by the amino acid sequence and/or the fatty acid-containing side chain used in the solid-phase synthesis.

TABLE 2
Amino acid sequences and modifications of polypeptides and derivatives thereof

| | SEQ | | | |
|---|---|---|---|---|
| | ID | | Site of | |
| ID | NO. | Amino acid sequence | modification | Fatty acid |
| D1 | SEQ | KCNTATCATQ RLAEFLRHSS | N terminus | C20 diacid + γGlu |
| | ID | NNFGPILPPT NVGSNTP-NH₂ | | |
| | NO. 1 | | | |
| D2 | SEQ | CSNLS TCVLG KLSQE LHKLQ | None | None |
| | ID | TYPRT NTGSG TP-NH₂ | | |
| | NO. 5 | | | |
| N1 | SEQ | KCNTATCATQ RLADFLRHSS | N terminus | C20 diacid + |
| | ID | NNLKPILPPT NVGSNT-trans-Hyp- | | 2*AEEAs |
| | NO. 2 | NH₂ | | |
| N2 | SEQ | KCNTATCATQ RLADFLRHSS | N terminus | C20 diacid + βAsp + |
| | ID | NNLKPILPPT NVGSNT-trans-Hyp- | | 2*AEEAs |
| | NO. 2 | NH₂ | | |
| N3 | SEQ | KCNTATCATQ RLADFLRHSS | N terminus | C18 diacid + βAsp + |
| | ID | NNLKPILPPT NVGSNT-trans-Hyp- | | 2*AEEAs |
| | NO. 2 | NH₂ | | |
| M1 | SEQ | KCNTATCATQ RLAEFLRHSS | N terminus | C18 diacid + γGlu |
| | ID | NNFGPILPPT NVGSNTP-NH₂ | | |
| | NO. 1 | | | |
| M2 | SEQ | KCNTATCATQ RLAEFLRHSS | N terminus | C20 diacid + |
| | ID | NNFGPILPPT NVGSNTP-NH₂ | | 2*AEEAs + γGlu |
| | NO. 1 | | | |
| M3 | SEQ | KCNTATCATQ RLAEFLRHSS | N terminus | C18 diacid + |
| | ID | NNFGPILPPT NVGSNTP-NH₂ | | 2*AEEAs + γGlu |
| | NO. 1 | | | |
| M5 | SEQ | ASQLS TAVLG RLSQE LHRLQ | None | None |
| | ID | DYPRT DVGSG SP-NH₂ | | |
| | NO. 3 | | | |
| M6 | SEQ | ASQLS TAVLG RLSDE LHRLQ | N terminus | C20 diacid + γGlu |
| | ID | DYPRT DVGSG SP-NH₂ | | |
| | NO. 4 | | | |
| M7 | SEQ | ASQLS TAVLG RLSDE LHRLQ | N terminus | C22 diacid + γGlu |
| | ID | DYPRT DVGSG SP-NH₂ | | |
| | NO. 4 | | | |
| M8 | SEQ | ASQLS TAVLG RLSDE LHRLQ | N terminus | C18 diacid + γGlu |
| | ID | DYPRT DVGSG SP-NH₂ | | |
| | NO. 4 | | | |
| M9 | SEQ | ASQLS TAVLG RLSDE LHRLQ | N terminus | C16 diacid + γGlu |
| | ID | DYPRT DVGSG SP-NH₂ | | |
| | NO. 4 | | | |
| M10 | SEQ | ASQLS TAVLG RLSDE LHRLQ | N terminus | C20 diacid + αGlu |
| | ID | DYPRT DVGSG SP-NH₂ | | |
| | NO. 4 | | | |
| M11 | SEQ | ASQLS TAVLG RLSDE LHRLQ | N terminus | C18 diacid + αGlu |
| | ID | DYPRT DVGSG SP-NH₂ | | |
| | NO. 4 | | | |
| M12 | SEQ | ASQLS TAVLG RLSDE LHRLQ | N terminus | C20 diacid + βAsp |
| | ID | DYPRT DVGSG SP-NH₂ | | |
| | NO. 4 | | | |
| M13 | SEQ | ASQLS TAVLG RLSDE LHRLQ | N terminus | C18 diacid + αAsp |
| | ID | DYPRT DVGSG SP-NH₂ | | |
| | NO. 4 | | | |
| M14 | SEQ | ASQLS TAVLG RLSDE LHRLQ | N terminus | C20 diacid + Trx |
| | ID | DYPRT DVGSG SP-NH₂ | | |
| | NO. 4 | | | |
| M15 | SEQ | ASQLS TAVLG RLSDE LHRLQ | N terminus | C18 diacid + Trx |
| | ID | DYPRT DVGSG SP-NH₂ | | |
| | NO. 4 | | | |
| M16 | SEQ | ASQLS TAVLG RLSDE LHRLQ | N terminus | C20 diacid + Inp |
| | ID | DYPRT DVGSG SP-NH₂ | | |
| | NO. 4 | | | |
| M17 | SEQ | ASQLS TAVLG RLSDE LHRLQ | N terminus | C18 diacid + Inp |
| | ID | DYPRT DVGSG SP-NH₂ | | |
| | NO. 4 | | | |
| M18 | SEQ | ASQLS TAVLG RLSDE LHRLQ | N terminus | C20 diacid + |
| | ID | DYPRT DVGSG SP-NH₂ | | γGlu + 2 AEEAs |
| | NO. 4 | | | |
| M19 | SEQ | ASQLS TAVLG RLSDE LHRLQ | N terminus | C20 diacid + |
| | ID | DYPRT DVGSG SP-NH₂ | | γGlu + 4 AEEAs |
| | NO. 4 | | | |
| M20 | SEQ | ASQLS TAVLG RLSDE LHRLQ | N terminus | C20 diacid + |
| | ID | DYPRT DVGSG SP-NH₂ | | γGlu + 6 AEEAs |
| | NO. 4 | | | |
| M21 | SEQ | ASQLS TAVLG RLSDE LHRLQ | N terminus | C20 diacid + |
| | ID | DYPRT DVGSG SP-NH₂ | | γGlu + 8 AEEAs |
| | NO. 4 | | | |
| M22 | SEQ | ASQLS TAVLG RLSDE LHRLQ | N terminus | C18 diacid + |
| | ID | DYPRT DVGSG SP-NH₂ | | γGlu + 2 AEEAs |
| | NO. 4 | | | |
| M23 | SEQ | ASQLS TAVLG RLSDE LHRLQ | N terminus | C20 diacid + |
| | ID | DYPRT DVGSG SP-NH₂ | | αGlu + 2 AEEAs |
| | NO. 4 | | | |
| M24 | SEQ | ASQLS TAVLG RLSDE LHRLQ | N terminus | C18 diacid + |
| | ID | DYPRT DVGSG SP-NH₂ | | αGlu + 2 AEEAs |
| | NO. 4 | | | |
| M25 | SEQ | ASQLS TAVLG RLSDE LHRLQ | N terminus | C20 diacid + |
| | ID | DYPRT DVGSG SP-NH₂ | | βAsp + 2 AEEAs |
| | NO. 4 | | | |
| M26 | SEQ | ASQLS TAVLG RLSDE LHRLQ | N terminus | C18 diacid + |
| | ID | DYPRT DVGSG SP-NH₂ | | αAsp + 2 AEEAs |
| | NO. 4 | | | |
| M27 | SEQ | ASQLS TAVLG RLSDE LHRLQ | N terminus | C20 diacid + Trx + 2 |
| | ID | DYPRT DVGSG SP-NH₂ | | AEEAs |
| | NO. 4 | | | |
| M28 | SEQ | ASQLS TAVLG RLSDE LHRLQ | N terminus | C18 diacid + Trx + 2 |
| | ID | DYPRT DVGSG SP-NH₂ | | AEEAs |
| | NO. 4 | | | |
| M29 | SEQ | ASQLS TAVLG RLSDE LHRLQ | N terminus | C20 diacid + Inp + 2 |
| | ID | DYPRT DVGSG SP-NH₂ | | AEEAs |
| | NO. 4 | | | |
| M30 | SEQ | ASQLS TAVLG RLSDE LHRLQ | N terminus | C18 diacid + Inp + 2 |
| | ID | DYPRT DVGSG SP-NH₂ | | AEEAs |
| | NO. 4 | | | |

N1-N3 or M1-M30 represent derivatives resulting from modifications of the amino acid sequences set forth in SEQ ID No. 1-SEQ ID No. 5 with the corresponding fatty acid-containing side chains in Table 2, or are polypeptides otherwise (where there is no fatty acid modification). The fatty acid-containing side chains are in the form of Z1+Z2+Z3, wherein Z1 is a C16-C22 fatty diacid; Z2 is selected from any one of γGlu, αGlu, βAsp, αAsp, Inp, and Trx, or is absent; Z3 is 0-8, for example 0, 1, 2, 3, 4, 5, or 6, AEEA(s); and Z1, Z2, and Z3 are linked via amide linkages. The C16-C22 fatty diacid moiety is located at the outer side of the fatty acid-containing side chain for modification relative to the amino acid sequence, and thus is positioned at the distal end of the linkage of the side chain to the amino acid sequence. For instance, M1 represents a derivative offered by modifying the amino acid sequence set forth in SEQ ID No. 1 with the fatty acid-containing side chain “C18 diacid+γGlu”, wherein the γGlu end of the side chain is linked to the amino acid sequence via a peptide bond and the C18 fatty diacid end is disposed at the distal end of the linkage of the side chain to the amino acid sequence.

### Example 2 Testing for Activity of the Polypeptide Derivatives In Vitro

The objective of this assay is to test the potency of the polypeptide derivatives in the present application on amylin and calcitonin receptors in vitro using a luciferase assay.

### 2.1 Amylin Receptor Agonism Testing

Using the standard protocol for construction of an amylin receptor/CRE-luc cell line, CHO-K1/Gα15/AMY3 cells (purchased from GenScript, with the calcitonin receptor and the receptor activity modifying peptide (RAMP) constructed already) were transfected with a plasmid containing a luciferase expression cassette driven by multiple copies of cAMP response elements (CREs). The cells were cultured in an F12 medium containing 200 μg/mL Zeocin, 2 μg/mL puromycin, 100 μg/mL hygromycin, and 400 μg/mL G418 to afford a stably transfected amylin receptor/CRE-luc cell line.

For the luciferase assay of amylin, the lyophilized powder obtained according to the method of Example 1 was dissolved in 20 mM phosphate buffer solution with a pH of 7.0, and diluted in a growth medium (F12 culture medium containing 10% FBS) to give a derivative sample with an initial concentration of 10 nM, which was then subjected to a 5-fold serial dilution in the growth medium to afford samples of 7 concentrations including 10 nM, 2 nM, 0.4 nM, etc. To each well of a white 96-well plate was added 50 μL of the tested sample solution at a respective concentration.

The stably transfected amylin receptor/CRE-luc CHO cells were resuspended in the growth medium at a certain density, and 50 μL of the cell suspension was added at a density of about 20,000 cells per well to the white 96-well plate containing the tested sample solution. After incubation of 24 hours at 37 °C and 5% CO₂, 100 μL of a Luciferase substrate was added to each well, incubated for 3 minutes, and finally was tested for luminescence on SpectraMax L (Molecular Devices) with the software SoftMax Pro 7.0.3 GxP. A standard curve was plotted using the fluorescent intensities for calculation of EC50.

Samples of compounds N2 and N3 were tested following the same method as the above, except that the tested sample solutions and the CHO cells were subjected to a condition where “after incubation of 4 hours at 37 °C and 5% CO₂, 100 μL of a Luciferase substrate was added to each well”.

Results were shown in the Table 3.

TABLE 3
Results for activity against the amylin receptor in cells

| ID | EC50/nM |
|---|---|
| D1 | 0.036 |
| M5 | 0.033 |
| M6 | 0.017 |
| M7 | 0.022 |
| M8 | 0.016 |
| M9 | 0.002 |
| M10 | 0.069 |
| M11 | 0.016 |
| M12 | 0.017 |
| M13 | 0.016 |
| M14 | 0.456 |
| M15 | 0.119 |
| M16 | 0.082 |
| M17 | 0.063 |
| M21 | 0.044 |
| M22 | 0.015 |
| M23 | 0.017 |
| M24 | 0.007 |
| M25 | 0.017 |
| M26 | 0.008 |
| M27 | 0.004 |
| M28 | 0.002 |
| M29 | 0.006 |
| M30 | 0.001 |
| N1 | 0.016 |
| N2 | 0.04 |
| / | / |
| / | / |
| N2 | 0.009 |
| N3 | 0.001 |

Data in Table 3 showed that the polypeptide derivatives of the present application had high activity in cells and exhibited great agonistic activity against the amylin receptor.

### 2.2 Calcitonin Receptor Agonism

### 2.2.1 Construction of a Calcitonin Receptor/CRE-Luc Cell Line

Using the standard protocol, CHO-K1/CALCR/Gα15 cells (purchased from GenScript, with the calcitonin receptor constructed already) were transfected with a plasmid containing a luciferase expression cassette driven by multiple copies of cAMP response elements (CREs). The cells were cultured in an F12 medium containing 200 μg/mL Zeocin, 2 μg/mL puromycin, and 100 μg/mL hygromycin to afford a stably transfected calcitonin receptor/CRE-luc cell line.

### 2.2.2 Luciferase Assay of Calcitonin

The lyophilized powder from Example 1 was dissolved in 20 mM phosphate buffer solution with a pH of 7.0, and diluted in a growth medium (F12 culture medium containing 10% FBS) to give a derivative sample with an initial concentration of 10 nM, which was then subjected to a 5-fold serial dilution in the growth medium to afford samples of 7 concentrations including 10 nM, 2 nM, 0.4 nM, etc. To each well of a white 96-well plate was added 50 μL of the tested sample solution at a respective concentration.

The stably transfected calcitonin receptor/CRE-luc CHO cells were resuspended in the growth medium at a certain density, and 50 μL of the cell suspension was added at a density of about 20,000 cells per well to the white 96-well plate containing the tested sample solution. After incubation of 24 hours at 37 °C and 5% CO₂, 100 μL of a Luciferase substrate was added to each well, incubated for 3 minutes, and finally was tested for luminescence on SpectraMax L (Molecular Devices) with the software SoftMax Pro 7.0.3 GxP. A standard curve was plotted using the fluorescent intensities for calculation of EC50. Results were shown in the following table.

TABLE 4
Results for activity against the calcitonin receptor in cells

| ID | EC50/nM |
|---|---|
| D1 | 0.086 |
| M6 | 0.028 |
| M7 | 0.194 |
| M8 | 0.165 |
| M9 | 0.120 |
| M11 | 0.071 |
| M12 | 0.073 |
| M13 | 0.085 |
| M14 | 0.587 |
| M15 | 0.469 |
| M16 | 0.376 |
| M17 | 0.259 |
| M18 | 0.069 |
| M25 | 0.138 |
| M24 | 0.081 |
| M26 | 0.024 |

Data in Table 4 showed that the polypeptide derivatives of the present application had high activity in cells and exhibited agonistic activity against the calcitonin receptor.

An integrated analysis of data of some polypeptide derivatives of the present application on activity against the amylin receptor (hAMY3R) and the calcitonin receptor (hCTR) in cells was shown in Table 5. Compared with a polypeptide derivative with a fatty acid-containing side chain in the form of Z1+Z2 (i.e., fatty diacid+amino acid), providing activity of the molecule D1 against hAMY3R and hCTR in cells as a benchmark, the polypeptide derivatives of the present application comprising a fatty acid-containing side chain in the form of Z1+Z2+Z3 (i.e., fatty diacid+amino acid+AEEA) showed a surprisingly significant difference in activity against the amylin and calcitonin receptors in cells and exhibited greater agonistic activity/relative activity against hAMY3R than hCTR, and such improvement in activity was especially pronounced when Z2 was Trx or Inp. For instance, M30 (SEQ ID NO. 4 modified with C18 diacid+Inp+2 AEEAs) had agonistic activity against the amylin receptor increased by nearly 70 folds as compared with M17 (SEQ ID NO. 4 modified with C18 diacid+Inp) and showed an about 42.5-fold increase in the ratio of activity against the amylin receptor to agonistic activity against the calcitonin receptor as opposed to the 1.7-fold increase achieved by M17 relative to the benchmark, displaying a stronger tendency for amylin receptor agonism.

Similarly, another derivative N3 comprising a different amino acid sequence (SEQ ID NO. 2) modified by the fatty acid-containing side chain in the form of Z1+Z2+Z3 (i.e., fatty

TABLE 5
Data on activity against the amylin receptor and the calcitonin receptor in cells

| | | | | Fold change | Fold change |
|---|---|---|---|---|---|
| | | | | in activity | in activity |
| | | hAMY3R | hCTR | against | against |
| ID | Fatty acid | EC50/nM | EC50/nM | hAMY3R | hCTR |
| D1 | C20 diacid + γGlu | 0.036 | 0.086 | 1.000 | 1.000 |
| M6 | C20 diacid + γGlu | 0.017 | 0.028 | 2.118 | 3.071 |
| M18 | C20 diacid + γGlu + 2 AEEAs | 0.016 | 0.069 | 2.25 | 1.246 |
| M12 | C20 diacid + βAsp | 0.017 | 0.073 | 2.118 | 1.178 |
| M25 | C20 diacid + βAsp + 2 AEEAs | 0.017 | 0.138 | 2.118 | 0.623 |
| M14 | C20 diacid + Trx | 0.456 | 0.587 | 0.079 | 0.147 |
| M27 | C20 diacid + Trx + 2 AEEAs | 0.004 | 0.533 | 9.000 | 0.161 |
| M16 | C20 diacid + Inp | 0.082 | 0.376 | 0.439 | 0.229 |
| M29 | C20 diacid + Inp + 2 AEEAs | 0.006 | 0.156 | 6.000 | 0.551 |
| M11 | C18 diacid + αGlu | 0.016 | 0.071 | 2.250 | 1.211 |
| M24 | C18 diacid + αGlu + 2 AEEAs | 0.007 | 0.081 | 5.143 | 1.061 |
| M15 | C18 diacid + Trx | 0.119 | 0.469 | 0.303 | 0.183 |
| M28 | C18 diacid + Trx + 2 AEEAs | 0.002 | 0.089 | 18.000 | 0.966 |
| M17 | C18 diacid + Inp | 0.063 | 0.259 | 0.571 | 0.332 |
| M30 | C18 diacid + Inp + 2 AEEAs | 0.001 | 0.101 | 36.000 | 0.848 |

### Example 3 Stability Study of the Polypeptide Derivatives

### 3.1 Materials and Devices

A stability test chamber (BINDER GmbH), a 0.01 mg-readability balance (METTLER TOLEDO), a pH meter (METTLER TOLEDO), a biosafety cabinet (ESCO), T2G-II small-sized motor-driven capping machine (Changsha zhongya pharmaceutical equipment co. LTD), a high-speed refrigerated centrifuge (Eppendorf), a sterilizer cabinet (Shinva Medical), Agilent 1260 high-performance liquid chromatographer, and Sepax Bio-C18 4.6*250 mm 3 μm 200 Å reversed phase column were used.

Reagents and consumables included: ultrapure water (18.2 MΩ, self-made), acetonitrile (HPLC grade), trifluoroacetic acid (HPLC grade), sodium dihydrogen phosphate (pharmaceutical grade, Hunan Jiudian Hongyang Pharmaceutical Co., Ltd), hydrochloric acid (pharmaceutical grade, Hunan Er-Kang Pharmaceutical Co., Ltd), sodium hydroxide (Analytical Reagent grade, Hushi®, Sinopharm Chemical Reagent Co., Ltd), vials, rubber stoppers, disposable syringes, and sterile filters.

### 3.2 Methods of the Assay

The lyophilized powder (1 mg/mL) of the polypeptide derivative prepared in accordance with the method of Example 1 was mixed with sodium dihydrogen phosphate (1.42 mg/mL) and dissolved in ultrapure water in the indicated mass-volume ratios. After adjusting pH to about 7.4 with hydrochloric acid/sodium hydroxide, the solution was filtered into a sterilized vial in a clean bench using a 0.22 μm sterile filter. The vial was then capped and placed into the stability test chamber at 40 °C. Detections were made on Days 7 (7d) and 26 (26d), and during the assay, changes in the characteristics and properties of the polypeptide were observed and recorded. The sample was then centrifuged for 3 min at 10,000 rpm at 4 °C, and the supernatant was transferred to a liquid phase sample vial for detecting the concentration and purity of the polypeptides using liquid phase chromatography described as below, wherein the concentration of the polypeptide=peak area/injection volume/extinction coefficient/60×flow rate, and the purity of the polypeptide=target peak area/total peak area×100%.

Conditions of the reversed phase chromatography Flow rate: 1.0 ml/min; Autosampler temperature: 15 °C; Column temperature: 25 °C; Detection wavelengths: 280 nm and 214 nm; Mobile phase A: 100% H₂O+0.05% TFA; Mobile phase B: 100% ACN; and Gradient of elution listed in Table 6:

TABLE 6
Listing of the gradient of elution
in the liquid phase chromatography

| Time (min) | 0 | 3 | 8 | 13 | 14 | 20 |
|---|---|---|---|---|---|---|
| B % | 25 | 25 | 70 | 70 | 25 | 25 |

### 3.3 Results of the Assay

As was observed in the heat accelerated stability assay, the solutions of the polypeptide derivatives of the present application remained to be clear on Day 7 of the heat accelerated assay, and exhibited distinctly higher clarity than the solution of the molecule D1 in which foreign matters were readily observable on Day 26 of the assay.

Table 7 demonstrated that there was no obvious change in concentration of the polypeptide derivatives provided in the present application during the heat accelerated stability assay under a neutral condition. In contrast to the molecule D2 showing a change of −40% in concentration on Day 7 of the accelerated testing, the present polypeptide derivatives underwent minimal changes in concentration, some of which (e.g., M6, M19, M22, M23, M24, M27, M29, and M30) even had a concentration change of less than 1% (denoted as “no change”). Concentration changes of solutions of the polypeptide derivatives of the present application remained below 10% even on Day 26 of the heat accelerated assay, and some were yet lower, standing at less than 3%.

TABLE 7
Concentration changes in the heat accelerated stability assay

| | Concen- | Concen- | | Concen- | Concen- |
|---|---|---|---|---|---|
| | tration | tration | | tration | tration |
| | change on | change on | | change on | change on |
| ID | 7 d/% | 26 d | ID | 7 d | 26 d |
| D2 | −40.064 | / | M18 | No change | −1.148 |
| M6 | No change | −1.865 | M19 | No change | −1.589 |
| M7 | No change | No change | M20 | No change | −3.662 |
| M8 | No change | −6.190 | M21 | −1.388 | −4.522 |
| M9 | −2.280 | −7.796 | M22 | 1.314 | −5.493 |
| M10 | −2.290 | −10.562 | M23 | −4.443 | −7.782 |
| M11 | −3.472 | −9.840 | M24 | No change | −3.954 |
| M12 | No change | −8.378 | M25 | −1.222 | −2.645 |
| M13 | −1.895 | −5.520 | M26 | −2.402 | −2.235 |
| M14 | −3.464 | −8.377 | M27 | No change | −2.286 |
| M15 | No change | −10.824 | M29 | No change | No change |
| M16 | −2.883 | −3.036 | M30 | −1.805 | −5.268 |
| M17 | −1.927 | −5.985 | | | |

Note:
concentration change = (concentration on 7 d or 26 d − concentration on 0 d)/concentration on 0 d × 100%

Table 8 revealed that there was no significant change in purity of the polypeptide derivatives provided in the present application during the heat accelerated stability assay under a neutral condition, as determined by the reversed phase chromatography. On Day 7 of the accelerated testing, purity changes of most of the polypeptide solutions were less than 1% when determined as above, or even no purity decrease was observed, and the polypeptide derivatives of the present application maintained a high purity whereas the molecule D1 in the prior art showed a purity decrease of 12.73% on Day 7 of the accelerated testing. Molecules such as M7, M18, M19, and M23 were further found to have nearly no noticeable change in purity even on Day 26 of the accelerated testing, showing great stability in purity.

TABLE 8
Purity changes in the heat accelerated stability assay
(Purity from the reversed phase chromatography-214 nm)

| | Purity | Purity |
|---|---|---|
| | change on | change on |
| ID | 7 d | 26 d |
| D1 | −12.73 | / |
| M6 | 1.64 | No change |
| M7 | No change | No change |
| M8 | No change | −3.3 |
| M9 | −1.67 | −4.82 |
| M10 | −1.66 | −5.98 |
| M11 | 1.01 | −2.93 |
| M12 | 1.12 | −1.96 |
| M13 | −1.15 | −3.76 |
| M14 | No change | −1.77 |
| M15 | No change | −5.67 |
| M16 | 3.42 | 2.18 |
| M17 | −1 | −3.9 |
| M18 | No change | No change |
| M19 | No change | No change |
| M20 | No change | −1.59 |
| M21 | −1.66 | −2.01 |
| M22 | 2.59 | −2.19 |
| M23 | No change | No change |
| M24 | −1.59 | −7.24 |
| M25 | −2.45 | −5.24 |
| M26 | −2.39 | −2.68 |
| M27 | No change | −1.27 |
| M29 | No change | −1.2 |
| M30 | No change | −4.34 |

Note:
purity change = purity on 7 d or 26 d − purity on 0 d

### Example 4 Results of Animal Study

SD (Sprague Dawley) rats weighing 200-250 g were used for this study. The rats were received at least 10-14 days before the study began such that they could accommodate to the experimental environment. After arrival, they were exposed to a reversed light/dark cycle (i.e., lights switched off in daytime and on in nighttime) for two weeks and were individually housed for the first week so as to ensure high data accuracy and test sensitivity. Throughout the acclimation and experiment period, the rats had free access to food and water. 5-8 rats were assigned to each testing group for the derivatives and were subcutaneously administered the derivatives at a dose of 10 nmol/kg or a vehicle (20 mM phosphate buffer, PB, pH 7.0), and the dosing timepoint was recorded for each group. After dosing, the rats were put back into cages where they were housed and could have access to food and water. Food consumption and weight change of the rats were recorded online or manually every 24 hours. Percent of weight change=(BWTₙ-BWT₀)/BWT₀*100%, wherein BW represented the value of body weight, and T₀ and Tₙ represented the time point of dosing and n hours post dose respectively. Dots in the figures were represented utilizing mean±standard error of the mean (SEM).

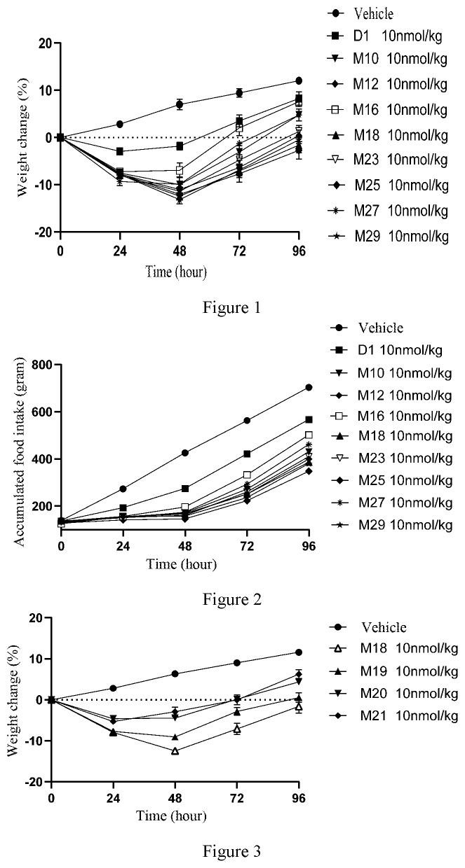

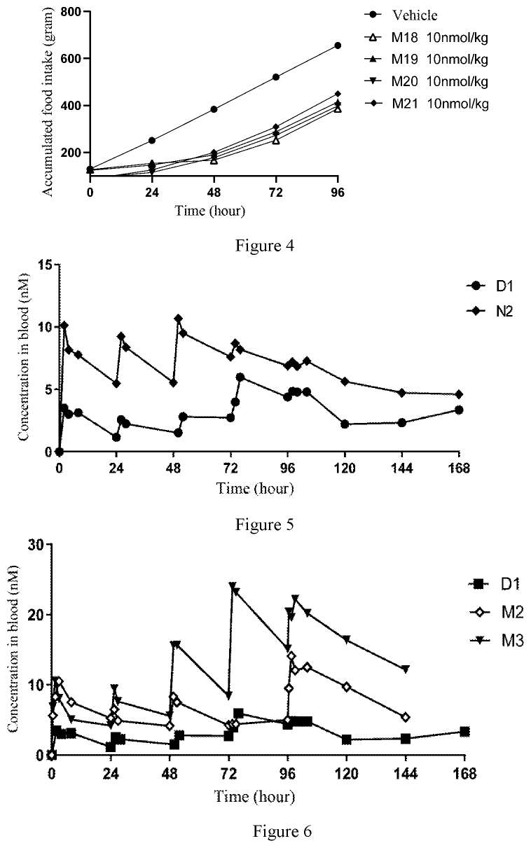

As shown in FIGS. 1-4, the polypeptide derivatives of the present application exerted an obviously superior effect on reducing both body weight and accumulated food intake in the SD rats as compared to the control (the vehicle or D1). More importantly, the addition of AEEA to the linker, e.g., in the polypeptide derivatives M18, M23, M25, M27, and M29, enabled apparently further reduced body weight and accumulated food intake in the SD rats as compared with a single amino acid linker in molecules with the same polypeptide structure.

### Example 5 Efficacy Assay in SD Rats-A Dose-Dependent Study

SPF male SD rats (7-8 weeks, 220-230 g) were employed in this study after health inspection and quarantine. The experimental animals were exposed to a room temperature of 20 °C-23 °C and a relative humidity of 40%-50%, and were provided with Co60 mouse reproduction feed 1035 and purified water supplied with water feeding bottles for free intake throughout the health inspection and quarantine and the study.

In the assay for efficacy comparison between compounds D1 and N3, the animals were assigned to 5 groups of n=5 based on their mean body weight, namely, the vehicle group (20 mM phosphate buffer, PB, pH 7.0), the compound D1 groups of varying doses (30 nmol/kg and 100 nmol/kg), and the compound N3 groups of varying doses (30 nmol/kg and 100 nmol/kg).

In the assay for efficacy comparison between compounds N2 and N3, the animals were assigned to 7 groups of n=5 based on their mean body weight, namely, the vehicle group (20 mM phosphate buffer, PB, pH 7.0), the compound N2 groups of varying doses (5 nmol/kg, 25 nmol/kg, and 125 nmol/kg), and the compound N3 groups of varying doses (5 nmol/kg, 25 nmol/kg, and 125 nmol/kg).

The animals were administered a single dose via subcutaneous injection in the study, wherein the day of administration was designated as Day 1/Time 0 (TO), the initial body weight of the animals were recorded and the initial feed was added on Day 0, and the body weight of the studied animals as well as the remaining food were recorded daily for calculating the percentage of weight change and the amount of food intake, any anomalies during which were required to be recorded and reported. During the study, the rats were in good condition and no anomaly was observed. Percent of weight change=(BWTₙ−BWT₀)/BWT₀*100%, wherein BW represented the value of body weight, and T₀ and Tₙ represented the time point of dosing and n hours post dose respectively. Dots in the figures were represented utilizing mean±standard error of the mean (SEM). Food intake was the amount of food consumed by each of the animals per day.

TABLE 9

| | 24 h weight | 24 h food |
|---|---|---|
| Group | change (%) | intake (g) |
| Vehicle | 2.095 | 25.000 |
| D1, 30 nmol/kg dose group | −6.416 | 5.250 |
| D1, 100 nmol/kg dose group | −9.103 | 2.100 |
| N3, 30 nmol/kg dose group | −8.422 | 2.100 |
| N3, 100 nmol/kg dose group | −11.276 | 0.500 |

TABLE 10

| | 24 h weight | 24 h food |
|---|---|---|
| Group | change (%) | intake (g) |
| Vehicle | 4.712 | 25.610 |
| N2, 5 nmol/kg dose group | 0.688 | 18.595 |
| N2, 25 nmol/kg dose group | −3.188 | 9.280 |
| N2, 125 nmol/kg dose group | −7.110 | 1.585 |
| N3, 5 nmol/kg dose group | −0.316 | 11.920 |
| N3, 25 nmol/kg dose group | −3.822 | 6.735 |
| N3, 125 nmol/kg dose group | −9.000 | 0.120 |

The compound N2 had a significantly greater effect on reducing body weight than D1 (see Example 3 in CN 202310614769.1). Table 9 showed that N3 also obviously exhibited an enhanced effect on reducing body weight and accumulated food intake in the SD rats in a dose-dependent manner as compared with D1. N2 and N3 are additional derivatives comprising a different amino acid sequence (SEQ ID NO. 2) modified by the fatty acid-containing side chain in the form of Z1+Z2+Z3 (i.e., fatty diacid+amino acid+AEEA), in the comparison of which N3 unexpectedly showed a stronger effect on reducing body weight and food intake of the animals (Table 10).

### Example 6 Study on PK Profile of Amylin Derivative Tablets

The amylin derivatives were formulated as oral tablets, which further comprise the same amount of the oral delivery agent PNAC with a structure of:

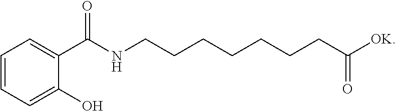

N-[8-(2-hydroxybenzoyl)amino]caprylic acid (NAC) was prepared utilizing the method described in Example 1 of the International Patent Application No. WO2008/028859. Isopropanol (22070.0 ml, 4.0 vol) was added to a 50 μL reactor and stirred, and then NAC (5518 g, 1.0 eq) was added. After the system was warmed to 50 °C, a 50% solution of potassium hydroxide (1304.0 g, 1.0 eq) was added dropwise, finally giving a clear yellow solution, which was allowed for reaction at 50 °C for 1 h. The reaction solution was then concentrated at 40 °C in portions to yield a light orange crude.

The combined crude was added to isopropanol (19310.0 ml, 3.5 vol), triturated for 1 h, and then subjected to suction filtration. The filter cake was rinsed with isopropanol (2760.0 ml, 0.5 vol), transferred to a vacuum dryer, pressure-balanced with nitrogen, dried for 16 h at 60 °C, and then transferred to a vacuum dryer again and dried for 24 h at 100 °C. After drying, a solid PNAC product of 4.52 kg in total was obtained as off-white powder with a yield of 72.8%.

Table 11 showed the amount of essential ingredients contained in tablets of the polypeptide derivatives comprising a fatty acid-containing side chain.

TABLE 11
An example of tablets of the polypeptide derivatives
prepared in the present application

| | | Delivery | Lubricant | |
|---|---|---|---|---|
| | API | agent | (magnesium | Total |
| | (polypeptide) | (PNAC) | stearate | mass |
| | Mass per | Mass per | for example) | Mass per |
| | tablet | tablet | Mass per | tablet |
| Formulation | (mg) | (mg) | tablet (mg) | (mg) |
| 1 | 7 | 300 | 8 | 315 |

The tablets of the polypeptide derivatives comprising a fatty acid-containing side chain were prepared by sieving the polypeptide derivatives and PNAC, mixing them with excipients uniformly, and performing direct compression. Male Beagle dogs aged 10-15 months (9-12 kg) were each orally administered amylin derivatives (D1 and N2) at a dose of 7 mg once daily for 5 consecutive days (n=5), with the first day of oral administration of the protein molecule-based drugs designated as Day 1, and the last day as Day 5. Whole blood of the animals was collected pre dose (−10 min) and 2 h, 4 h, and 8 h post dose on Day 1, pre dose (-10 min) and 2 h and 4 h post dose on Day 2-Day 4, and pre dose (−10 min) and 2 h, 4 h, 8 h, 24 h, 48 h, and 72 h post dose on Day 5, and processed into plasma using heparin sodium as an anticoagulant. The plasma was stored at −80 °C for subsequent analysis of the plasma level of APIs using LC-MS/MS (Waters ACQUITY I Class Premier UPLC tandem with Sciex 6500+QQQ), wherein the charge-to-mass ratio of the ion pair for D1 was 1102.9/1074.5, and that for N2 was 945.6/919.8. Experimental data was plotted using GraphPad Prism9.3.1.

The results were shown in FIG. 5, demonstrating that compared with the tablet comprising the compound D1, the molecule of the compound N2 showed a special synergy with the delivery agent PNAC in the tablet, exhibiting an extraordinarily high oral bioavailability.

In the comparison of oral bioavailability between D1 and N3, N3 and the delivery agent PNAC in the tablet also produced the synergistic effect of significantly improving the bioavailability.

### Example 7 Study on Influence of Side Chain Modification on PK Profile of Oral Tablets of the Polypeptide Derivatives

Additional polypeptide analogs of amylin comprising a different sequence were used to further verify the influence of side chain modification on oral bioavailability of the polypeptide derivatives. Oral tablets of polypeptide derivatives D1, M2, and M3 shown in Table 1, which only differed by the side chain, were tested for their PK profile using a similar method to that described in Example 6.

The results were shown in FIG. 6, revealing that the polypeptide derivatives M2 and M3 enabled apparently higher absorption than the molecule D1 after oral administration, which was similar to the result in Example 6 and further indicated that modification with the side chain structures provided in the present application contributed effectively to improvement in oral bioavailability of the polypeptide derivatives, and M3 containing a C18 fatty acid had higher oral bioavailability than M2 containing a C20 fatty acid.

Although embodiments of the present application have been described as above, the present application is not limited to the above specific embodiments, which are only illustrative and directory but not restrictive, and application fields. Without departing from the scope of the claims of the present application, many variants may occur to those of ordinary skill in the art with inspiration from this specification.

1. A derivative of a polypeptide, consisting of the polypeptide and a side chain linked thereto in the form of Z1+Z2+Z3, wherein the polypeptide consists of the amino acid sequence KCNTATCATQ RLADFLRHSS NNLKPILPPT NVGSNT-trans-Hyp-NH₂ (SEQ ID NO: 2), and wherein Z1 is a C18 fatty diacid; Z2 is β-aspartic acid; and Z3 comprises 2 AEEAs, wherein AEEA has the structure:

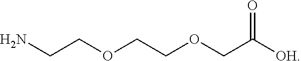 [SMILES: NCCOCCOCC(=O)O]

2. The derivative of the polypeptide of claim 1, wherein Z1, Z2, and Z3 are linked via amide linkages.

3. The derivative of the polypeptide of claim 1, wherein the side chain is linked to the N-terminus of the polypeptide.

4. A pharmaceutical composition comprising the derivative of claim 1 or a pharmaceutically acceptable salt thereof, and a pharmaceutically acceptable excipient.

5. The pharmaceutical composition of claim 4, wherein the composition comprises one or more oral delivery agents.

6. The pharmaceutical composition of claim 5, wherein the oral delivery agent comprises a salt of N-[8-(2-hydroxybenzoyl)amino]caprylic acid (NAC).

7. The pharmaceutical composition of claim 5, wherein the oral delivery agent comprises PNAC

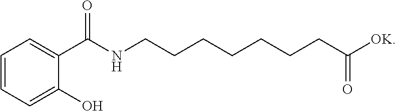

8. A method of preventing and/or treating overweight and/or obesity and/or type I or type II diabetes and/or osteoporosis and/or neuropathic pain, comprising administering to a subject a prophylactically or therapeutically effective amount of the derivative of claim 1.

9. A method of preventing and/or treating overweight and/or obesity and/or type I or type II diabetes and/or osteoporosis and/or neuropathic pain, comprising administering to a subject a prophylactically or therapeutically effective amount of the pharmaceutical composition of claim 4.

10. A method of reducing food intake, comprising administering to a subject an effective amount of the derivative of claim 1.

11. A method of reducing food intake, comprising administering to a subject an effective amount of the pharmaceutical composition of claim 4.
